(19) United States
(12) Patent Application Publication
(10) Pub. No.: US 2023/0257828 A1
(43) Pub. Date: Aug. 17, 2023

(54) ADM2 GENE MARKER FOR DIAGNOSIS OR PROGNOSIS PREDICTION OF THYROID CANCER AND USES THEREOF

(71) Applicant: The Industry & Academic Cooperation in Chungnam National University (IAC), Daejeon (KR)

(72) Inventors: Minho Shong, Daejeon (JP); Yea Eun Kang, Daejeon (CN)

(21) Appl. No.: 18/296,763

(22) Filed: Apr. 6, 2023

Related U.S. Application Data
(63) Continuation of application No. 16/445,322, filed on Jun. 19, 2019, now abandoned.

(30) Foreign Application Priority Data
Jan. 22, 2019 (KR) 10-2019-0007842

(51) Int. Cl. C12Q 1/6886; G01N 33/574

(52) U.S. Cl. CPC C12Q 1/6886; G01N 33/57407; C12Q 2600/106; C12Q 2600/158; G01N 2333/5753; C12Q 2600/118

(57) ABSTRACT

The present invention relates to a composition for diagnosis or prognosis prediction of thyroid cancer, which includes an agent for measuring the expression level of mRNA of ADM2 gene or a protein thereof, a kit for diagnosis or prognosis prediction of thyroid cancer, which includes the composition, and a method for providing information for diagnosis or prognosis prediction of thyroid cancer using the composition or the kit.

### CROSS-REFERENCE TO RELATED APPLICATIONS

This application is a continuation-in-part (CIP) of application Ser. No. 16/445,332 filed on Jun. 19, 2019, which claims priority to and the benefit of Korean Patent Application No. 10-2019-0007842 filed in the Korean Intellectual Property Office on Jan. 22, 2019, the entire contents of which are incorporated herein by reference.

### TECHNICAL FIELD

The present invention relates to ADM2 (adrenomedullin 2) gene marker for diagnosis or prognosis prediction of thyroid cancer and uses thereof.

### BACKGROUND ART

The thyroid is a butterfly-shaped organ located in the lower portion of the thyroid cartilage and located in front of the airway, which is the air passage when breathing and functions to produce and store the thyroid hormone and to send it to the organ that needs it.

Thyroid cancer is a generic term for cancer which occurs in the thyroid, which is the most common malignancy of the endocrine system. Thyroid cancer is generally divided into “well-differentiated thyroid cancer” and “other thyroid cancer,” which is classified into papillary carcinoma, follicular carcinoma, medullary thyroid carcinoma, and anaplastic carcinoma (undifferentiated thyroid cancer) according to the histological shape, the origin cells of cancer and the differentiation degree of cancer. Among these, differentiated tumors such as thyroid papillary carcinoma and thyroid follicular carcinoma have a good prognosis in general but their survival rate is rapidly lowered when invading into surrounding tissues or metastasizing to other organs. Anaplastic carcinoma is a rare but notorious undifferentiated cancer in which most patients may die within six months. Thyroid cancer is associated with genetic alterations, e.g., BRAF^{V600E}, which may cause carcinomatous changes in hormone-secreting epithelial cells. Epidemiological studies have shown that overnutrition is related to the development and progression of cancer.

Excessive nutrients have led to an increase in obesity-related metabolic diseases and are associated with an increased risk and mortality from cancer. Obesity is a particularly strong risk factor for colon, renal, pancreatic, endometrial, and breast cancer. There is a link between obesity and thyroid cancer. Although epidemiological studies have shown that obesity increases the incidence of thyroid cancer, it remains unclear how obesity stimulates the development of or influences the prognosis of this cancer type. The obesity-cancer connection mechanisms include anabolic hormonal signalling, systemic inflammation, and adipocyte-cancer crosstalk. Insulin and insulin-like growth factor levels are increased in obesity and may provide a critical hormonal signal that mediates the proliferation and survival of thyroid cancer. Obesity is also associated with chronic systemic inflammation. Tumour necrosis factor-alpha and interleukins (IL-10, IL-6, and IL-8), the expression of which are also elevated in obesity, are linked to thyroid cancer initiation and progression. Adipocytes participate in crosstalk with parenchymal cells to promote cancer initiation and progression. Adipocytes potentiate tumour cell proliferation and invasion in multiple cancers in vitro and in vivo. However, the mechanisms underlying the obesity-thyroid cancer connection remains unclear.

Most thyroid cancer can be cured by the first surgery. Referring to disease stage, 80% to 85% of patients with disease stage are at low risk of death. However, some of them have a high-risk factor for recurrence. In fact, it has been reported that 20% of patients recurred and at most 50 to 60% of patients may die. Since it is more difficult to determine the prognosis after a recurrence of thyroid cancer than in the first operation, there are many studies on methods to reduce recurrence by analyzing factors related to the thyroid cancer's recurrence, and the recurrence frequency, recurrence site, disease-free period, recurrence treatment, treatment outcome and the like.

Meanwhile, Korean Patent No. 1587635 discloses a method for detecting methylation of a thyroid cancer-specific methylation marker gene for diagnosis of thyroid cancer, and Korean Patent No. 1845590 discloses a composition for predicting the prognosis of lung cancer using a gene. However, the ADM2 gene marker for diagnosis or prognosis prediction of thyroid cancer of the present invention and use thereof has not been described.

### SUMMARY OF THE INVENTION

The present invention has been made by the above demands, and the present inventors have performed RNA sequence analysis on a control group without cancer, an animal model of thyroid cancer under a normal chow diet, and an animal model of thyroid cancer under high-fat diet in order to confirm the mechanism of the clinical relevance of obesity and thyroid cancer. As a result of the analysis, it was confirmed that the expression level of ADM2 (adrenomedullin 2) gene was significantly different in the above three groups. In particular, the expression level of ADM2 gene in the animal model was increased in proportion to the size of thyroid cancer. Clinical sample analysis indicated that the expression of ADM2 protein in the obese group with thyroid cancer was increased compared to the normal weight group with thyroid cancer, and the expression of ADM2 protein in the obese group with recurrent thyroid cancer was significantly increased. The present inventors confirmed the possibility of the ADM2 gene or its protein as a biomarker for diagnosis or prognosis prediction of thyroid cancer, thereby completing the present invention.

In order to achieve the objectives, the present invention provides a composition for diagnosis or prognosis prediction of thyroid cancer, the composition including an agent for measuring the expression level of mRNA of ADM2 (adrenomedullin 2) gene or its protein.

The present invention provides a kit for the diagnosis or prognosis prediction of thyroid cancer, the kit including the composition.

The present invention provides a method for providing information for the diagnosis of thyroid cancer by measuring the expression level of mRNA of the ADM2 gene or protein expressed from the gene from a biological sample isolated from a suspected thyroid cancer patient.

The present invention provides a method for providing information for prognosis prediction of thyroid cancer by measuring the expression level of mRNA of the ADM2 gene or protein expressed from the gene from a biological sample isolated from obese or recurrent patients with thyroid cancer.

The present invention provides a method of screening a therapeutic agent for thyroid cancer, the method including measuring the expression level of ADM2 gene or protein encoded by the gene after administering the candidate substance expected to be capable of curing the thyroid cancer.

The ADM2 gene of the present invention or protein thereof is a novel biomarker for the diagnosis, prognosis, or recurrence prediction of thyroid cancer, which can be useful for diagnosing thyroid cancer or predicting the severity of the disease and can be useful for screening therapeutic agents for thyroid cancer through analysis of expression level changes of the ADM2 gene or protein thereof.

The present invention showed that ADM2 is released from cancer cells under mitochondrial stress resulting from overnutrition and acts as a secretory factor determining the progressive properties of thyroid cancer.

The present invention identified the cell non-autonomous factor responsible for the progression of BRAF^{V600E} thyroid cancer under overnutrition conditions and presented a mouse model for inducible thyrocyte-specific activation of BRAF^{V600E}, which showed features similar to those of human papillary thyroid cancer. LSL-Braf^{V600E}; TgCreER^{T2} showed thyroid tumour development in the entire thyroid, and the tumour showed more abnormal cellular features with mitochondrial abnormalities in mice fed a high-fat diet (HFD).

Transcriptomics revealed that ADM2 was increased in LSL-Braf^{V600E}; TgCreER^{T2} mice fed HFD. ADM2 was upregulated on the addition of a mitochondrial complex I inhibitor or palmitic acid with integrated stress response (ISR) in cancer cells. ADM2 stimulated protein kinase A (PKA) and extracellular signal-regulated kinase (ERK) in vitro. The knockdown of ADM2 suppressed the proliferation and migration of thyroid cancer cells.

The present invention showed that increased ADM2 expression was associated with ISR and poor overall survival. Consistently, upregulated ADM2 expression in tumour cells and circulating ADM2 molecules were associated with aggressive clinicopathological parameters, including body mass index, in thyroid cancer patients. ADM2 is released from cancer cells under mitochondrial stress resulting from overnutrition and acts as a secretory factor determining the progressive properties of thyroid cancer.

Parts of the present invention are described in Jung Tae Kim, Mi Ae Lim, Seong Eun Lee, Hyun Jung Kim, Hyun Yong Koh, Jung Ho Lee, Sang Mi Jun, Jin Man Kim, Kun Ho Kim, Hyo Shik Shin, Sun Wook Cho, Koon Soon Kim, Yea Eun Kang, Bon Seok Koo, Minho Shong, “Adrenomedullin2 stimulates progression of thyroid cancer in mice and humans under nutrient excess conditions” (the Journal of Pathology, Vol. 258 (3), pages 264-277), which is incorporated herein in its entirety by reference.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIGS. 1A, 1B, 1C, and 1D illustrate the results of RNA sequence analysis of gene expression levels in tissues of a control group without cancer (control), an animal model of thyroid cancer under a normal chow diet (NCD), and an animal model of thyroid cancer under high-fat diet (HFD).

FIG. 2 illustrates the results of confirming the expression of AMD2 protein in NCD and HFD by immunohistochemistry and a graph showing the ratio of AMD2-positive cells.

FIGS. 3A, 3B, and 3C illustrate the results of confirming the expression of AMD2 protein in the thyroid carcinoma tissues of thyroid cancer patients with normal body weight (body mass index<25) (A), thyroid cancer patients with obesity (body mass index>25) (B), and recurrent thyroid cancer patients with obesity (C) by immunohistochemistry.

FIG. 4 illustrates LSL-Braf^{V600E}; TgCreER^{T2} mice developed a solid mass region with papillary growing with dysmorphic and variable follicles on the administration of tamoxifen: (A) Diagram of the experimental design. Tamoxifen was injected into LSL-Braf^{V600E} mice and LSLBraf⁶⁰⁰E; TgCreER^{T2} mice at 8 weeks of age to induce Braf^{V600E} knock-in. All mice were sacrificed at 22 weeks of age. (B) Body weight of LSL-Braf^{V600E} mice and LSLBraf^{V600E}; TgCreER^{T2} mice at 22 weeks of age (LSL-BraJf⁶⁰⁰E mice, n=8; LSLBraf^{V600E}; TgCreER^{T2}, n=3). (C) Representative gross image of thyroid glands in LSL-Braf^{V600E} mice and LSL-Braf^{V600E}; TgCreER^{T2} mice. (D) Thyroid weight of LSL-Braf^{V600E} mice and LSLBraf^{V600E}; TgCreER^{T2} mice (LSL-Braf^{V600E} mice, n=7; LSL-Braf^{V600E}; TgCreER^{T2}, n=3). (E) Histological images of the thyroid gland in LSL-Braf^{V600E} mice and LSL-Braf^{V600E}; TgCreER^{T2} mice. (F) Immunoblot analysis of BRAF^{V600E}, CyclinD1, PCNA, and GAPDH in thyroid gland from LSL-Braf^{V600E} mice and LSL-Braf^{V600E}; TgCreER^{T2} mice (LSL-Braf^{V600E} mice, n=8; LSL-Braf^{V600E}; TgCreER^{T}2, n=3). Mean±SEM. *p<0.05 or **p<0.01 or ***p<0.001 (two-tailed Student's t-test).

FIG. 5 illustrates diet-induced obesity causes tumour progression in LSL-Braf^{V600E}. TgCreER^{T2} mice: (A) Diagram of the experimental design. Tamoxifen was injected at 8 weeks of age. CD and HFD were fed for 12 weeks starting at 10 weeks of age. All mice were sacrificed at 22 weeks of age. (B) Body weight of LSL-Braf^{V600E}; TgCreER^{T2} mice fed CD or HFD at 22 weeks of age (LSL-Braf^{V600E}; TgCreER^{T2} mice fed CD, n=3; SL-Braf^{V600E}; TgCreER^{T2} mice fed HFD, n=5). (C) Glucose tolerance test at 20 weeks of age. Glucose (1 g/kg) was administered, and blood glucose was measured at 0, 15, 30, 45, and 60 min (n=4). (D) Serum concentration of thyroid hormones (free T3 and free T4) (LSL-Braf^{V600E}; TgCreER^{T2} mice fed CD, n=3; SL-Braf^{600E}; TgCreER^{T2} mice fed HFD, n=5). (E) Serum concentration of TSH (LSL-Braf^{V600E}; TgCreER^{T2} mice fed CD, n=3; SL-Braf^{V600E}; TgCreER^{T2} mice fed HFD, n=4). (F) Thyroid images and weight of LSL-Braf^{V600E}; TgCreER^{T2} mice fed CD or HFD at 22 weeks of age (LSL-Braf^{600E}; TgCreER^{T2} mice fed CD, n=3; SL-Braf^{V600E}; TgCreER^{T2} mice fed HFD, n=5). (G) Most enlarged region of cancer foci and tumour foci/total area, black lines indicate tumour foci (n=4). (H) Immunohistochemistry images and graph on thyroid cancer tissue (n=4). (I) Immunoblot analysis of BRAF^{V600E}, CyclinD1, p-ERK, ERK, and α-tubulin of the thyroid gland from LSL-Braf^{V600E}; TgCreER^{T2} mice fed CD or HFD (n=3). Mean±SEM. *p<0.05 or **p<0.01 or ***p<0.001 (two-tailed Student's t-test).

FIG. 6 illustrates transcriptome analyses revealed that ADM2 is upregulated by metabolic stress in the thyroid gland of LSL-Braf^{V600E}; TgCreER^{T2} mice: (A) Volcano plot and heat map of RNAseq analysis from LSL-Braf^{V600E}; TgCreER^{T2} mice fed CD or HFD (LSL-Braf^{600E}; TgCreER^{T2} mice fed CD, n=2; SL-Braf^{V600E}; TgCreER^{T2} mice fed HFD, n=3). (B) RT-qPCR analysis of ADM2 mRNA expression in thyroid gland from LSL-Braf^{V600E}; TgCreER^{T2} mice fed CD or HFD (n=5). (C) Immunoblot analysis of ADM2, ATF4, DDIT3, and β-actin of the thyroid gland from LSL-Braf^{V600E}; TgCreER^{T2} mice fed CD or HFD (n=3) (D) Representative immunohistochemistry images of ADM2 expression in thyroid gland from LSL-Braf^{V600E}. TgCreER^{T2} mice fed CD or HFD; scale bars: 50 m (n=4) (E) Electron microscopy analysis of thyrocytes from LSL-Braf^{V600E}; TgCreER^{T2} mice, black arrows indicate mitochondria; scale bars, 500 nm (n=5). Mean±SEM. *p<0.05 or **p<0.01 or ***p<0.001 (two-tailed Student's t-test).

FIG. 7 illustrates that metabolic stress induces the expression of ADM2 and the activation of ISR signalling pathways in the thyroid cancer cell line: (A) ADM2 expression in thyroid cell lines (B) RT-qPCR analysis for ADM2 mRNA in thyroid cancer cell lines in the presence or absence of rotenone (1 μM) for 24 h (n=3). (C) RT-qPCR analysis for ISR and mitokine genes of thyroid cancer cell lines in the presence or absence of rotenone (1 μM) (n=3). (D) Immunoblot analysis of ADM2 and ISR-related proteins of thyroid cancer cell line in the presence or absence of rotenone (1 μM). (E) Cell viability test using a wst-8 assay of a thyroid cancer cell line after 24 h treatment of palmitic acid (0, 25, 50, 100 μM) (n=5). (F) Oxygen consumption rates (OCR) of thyroid cancer cell lines after 24 h treatment with palmitic acid (0, 25, and 50 μM) (8505 C, n=4; BCPAP, n=5). CCCP: carbonyl cyanide m-chlorophenyl hydrazone. (G) RT-qPCR analysis for ADM2 mRNA in thyroid cancer cell lines treated with 50 M palmitic acid for 24 h (n=3). (H) RT-qPCR analysis for ISR and mitokine genes in a thyroid cancer cell line treated with 50 μM palmitic acid for 24 h (n=3). (I) Immunoblot analysis for ADM2 and ISR-related proteins from thyroid cancer cell lines treated with 50 μM palmitic acid for 24 h. Mean±SEM. *p<0.05 or **p<0.01 or ***p<0.001 (two-tailed Student's t-test).

FIG. 8 illustrates that ADM2 deficiency slows cell proliferation and migration of thyroid cancer cell lines through ERK signalling: (A) Immunoblot analysis of recombinant AMD2-induced PKA and ERK signal (n=3). (B) Proliferation analysis in the thyroid cancer cell line transfected with shADM2 (n=3). (C) Migration analysis in the thyroid cancer cell line transfected with shADM2 (n=3). (D) Immunoblot analysis for ADM2, AKT, ERK, STAT3, and NF-kB in the thyroid cancer cell lines transfected with shADM2. Mean±SEM. *p<0.05 or **p<0.01 or ***p<0.001 (two-tailed Student's t-test).

FIG. 9 illustrates that ADM2 is related to the ISR and poor clinicopathological features in the human thyroid: (A, B) Correlation analysis of ADM2 with ISR genes (A) GTEx-Thyroid gland (B) TCGA-THCA. (C-E) Comparison of groups divided using ADM2 expression on quartile in TCGA-THCA. (C) The violin and volcano plots. (D) Overall survival and disease-specific survival. (E) Bar plots representing the gene set enrichment analysis of the Gene-Ontology biological process. (F, G) (F) Comparisons in CNUH cohort and (G) Immunoblot analysis for ADM2 in the thyroid gland of PTC patients. Serum level of ADM2 in PTC patients divided by clinicopathological features. Mean±SEM. *p<0.05 or **p<0.01.

In the figures, reference numbers refer to the same or equivalent parts of the present invention throughout the several figures of the drawing.

### DETAILED DESCRIPTION

Hereinafter, exemplary embodiments of the present invention will be described in detail with reference to the accompanying drawings.

In order to accomplish the object of the present invention, the present invention provides a composition for diagnosis or prognosis prediction of thyroid cancer, the composition including an agent for measuring the expression level of mRNA of ADM2 (adrenomedullin 2) gene or its protein.

ADM2 gene information is registered in the National Center for Biotechnology Information (NCBI) (NC_000022.11), but the relationship between ADM2 gene and thyroid cancer has not been known at all.

The ADM2 gene of the present invention may preferably include the nucleotide sequence represented by SEQ ID NO: 1, but is not limited thereto. Further, homologs of the nucleotide sequences are included within the scope of the present invention. In particular, the gene includes a nucleotide sequence having a sequence homology of 70% or more, preferably 80% or more, more preferably 90% or more, and most preferably 95% or more, respectively, with the nucleotide sequence represented by SEQ ID NO: 1. “% of sequence homology” to polynucleotides is identified by comparing two optimally aligned sequences by the comparison region, and a portion of the polynucleotide sequence in the comparison region may include addition or deletion (i.e., gap) compared with the reference sequence (without addition or deletion) for the optimal alignment of the two sequences.

In the present invention, the term “diagnosis” is to identify the presence or characteristic of a pathological condition. For the purposes of the present invention, the diagnosis is to ascertain whether or not thyroid cancer has developed.

In the present invention, the term “prognosis” means an increase or decrease in thyroid cancer severity. Prognosis in cancer patients generally refers to metastasis or survival time within a period of time after the cancer onset or surgery. The prognosis prediction shows the direction of future treatment, in particular, the chemotherapy of the thyroid cancer patients. It can be interpreted as a kind of all the act that predicts the progress after the treatment by comprehensively considering the physiological or environmental condition of the patient after treatment.

Accordingly, for the purpose of the present invention, the prognosis prediction means predicting whether the disease is warned and completely cured after the thyroid cancer treatment, thereby predicting the disease-free survival rate or the survival rate of the thyroid cancer patient. For example, the prediction of “good prognosis” implies that the disease-free survival rate or survival rate of the patient after the thyroid cancer treatment is high and the possibility of treating the thyroid cancer patient is high, and the prediction of “poor prognosis” implies that the disease-free survival rate or survival rate of the patient after the thyroid cancer treatment is low so that there is a high probability of recurrence from thyroid cancer patients and death due to thyroid cancer. The disease-free survival rate means the possibility that the patient can survive without recurrence of cancer after the thyroid cancer treatment, and the survival rate means the possibility that the patient can survive regardless of the recurrence of cancer after the thyroid cancer treatment.

In the present invention, the term “mRNA expression level measurement” is a process for confirming the presence and expression level of the mRNA of the thyroid cancer marker gene (ADM2) in a biological sample for the diagnosis or prognosis prediction of thyroid cancer. To this end, the analysis method includes reverse transcriptase polymerase chain reaction (RT-PCR), competitive RT-PCR, quantified real-time PCR, real-time quantitative RT-PCR, RNase protection assay (RPA), Northern blotting, DNA chip, and the like, but is not limited thereto.

The agent for measuring the mRNA expression level of the ADM2 gene in the present invention may preferably include, but not limited to, a primer, a probe, or an antisense oligonucleotide. The primer, probe, or antisense oligonucleotide may be designed using methods, programs or tools known to those skilled in the art by reference to the nucleotide sequence of the ADM2 gene (the accession number NM_001253845.1).

In the present invention, the term “primer” means a short nucleic acid sequence having a free 3′ hydroxyl group, which is able to form a base pair with a complementary template, and functions as a starting point for amplifying the template strands. The primer can initiate DNA synthesis in the presence of a reagent for polymerization in a suitable buffer solution, at a suitable temperature (i.e., DNA polymerase, or reverse transcriptase) and four different nucleoside triphosphates. In the present invention, PCR amplification can be performed using a sense and antisense primer capable of binding to the base sequence of the ADM2 gene to diagnose or predict the prognosis of thyroid cancer by the production of the desired product. PCR conditions and length of sense and antisense primers can be modified on the basis of the methods known in the art.

In the present invention, the term “probe” means a fragment of nucleic acid such as RNA or DNA, which is several to hundreds of bases capable of specifically binding to mRNA, and is labeled to identify the presence of specific mRNA. The probe can be prepared in the form of oligonucleotide probe, single-stranded DNA probe, double-stranded DNA probe, RNA probe, or the like. In the present invention, hybridization is performed using a probe complementary to the ADM2 polynucleotide, and then thyroid cancer can be diagnosed, or its prognosis can be predicted by the hybridization result. Selection of suitable probe and hybridization conditions can be modified on the basis of the methods known in the art.

In the present invention, the term “measuring the expression level of protein” is a process for confirming the presence and expression level of a protein expressed in a thyroid cancer marker gene (ADM2) from a biological sample for the diagnosis or prognosis prediction of thyroid cancer and may be performed by generally identifying the protein amount. Analysis methods for this may include, but not limited to, western blot, enzyme-linked immunosorbent assay (ELISA), radioimmunoassay (RIA), radioimmunodiffusion, Ouchterlony immunodiffusion, rocket immunoelectrophoresis, immunohistochemistry, immunoprecipitation assay, complement fixation assay, fluorescence activated cell sorting (FACS), protein chips and the like.

In the present invention, the agent for measuring the expression level of the protein is not limited thereto, but may preferably be an antibody or an aptamer that specifically binds to the ADM2 protein.

In the present invention, the term “antibody” is a term known in the art and refers to a specific protein molecule that indicates an antigenic region. With respect to the objects of the present invention, the antibody refers to an antibody that specifically binds to ADM2 protein, the marker of the present invention. The full length or a part of the ADM2 gene is cloned into an expression vector according to a conventional method so as to obtain a protein encoded by the full length or a part of the ADM2 gene and the resulting protein is used as an immunogen (antigen) so that the antibody can be prepared by the conventional method. There is no limitation in the form of the antibody of the present invention, and a polyclonal antibody, a monoclonal antibody, or a functional fragment thereof having antigen-binding property is also included, and all immunoglobulin antibodies are included. Furthermore, the antibody of the present invention also includes special antibodies, such as a humanized antibody. The functional fragment of the antibody molecule refers to a fragment having at least an antigen-binding function and includes Fab, F(ab′) 2, F(ab′)2, ScFv, and the like.

In the present invention, the term “aptamer” refers to a nucleic acid capable of strongly binding specifically to a specific molecule while maintaining a stable tertiary structure. It is compared with antibodies because of its specific binding function and has been evaluated as an alternative technology for antibodies.

The present invention provides a kit for diagnosis or prognosis prediction of thyroid cancer, the kit including the composition as described above.

The kit of the present invention may diagnose thyroid cancer or predict the prognosis of thyroid cancer by identifying the detection of the mRNA of the ADM2 gene or the protein expressed therefrom. The kit for diagnosis or prognosis prediction of thyroid cancer of the present invention includes an agent (for example, a primer or a probe) for detecting the expression level of the ADM2 gene or an agent (for example, an antibody or a aptamer) capable of specifically detecting a protein as well as one or more other component compositions, solutions or devices suitable for the analysis methods.

In the present invention, the kit to assess the mRNA expression level of ADM2 gene may be a kit that includes essential elements required for performing RT-PCR. An RT-PCR kit may include test tubes or other suitable containers, reaction buffers (varying in pH and magnesium concentrations), deoxynucleotides (dNTPs), enzymes such as Taq-polymerase and reverse transcriptase, Dnase, Rnase inhibitor, DEPC water, and sterile water, in addition to each pair of primers specific to the marker gene. It may include a pair of primers specific to the gene used as a quantitative control.

The kit of the present invention may also be a kit for diagnosis or prognosis, including essential elements required for performing a DNA chip. The DNA chip may include a base plate, onto which cDNAs corresponding to genes or fragments thereof are attached as a probe and reagents, preparations, enzymes, and the like for producing a fluorescent-labeled probe. Further, the base plate may also include a quantitative control gene and cDNA corresponding to fragments thereof.

When the kit of the present invention may be a kit for measuring the expression level of ADM2 protein, it may include a matrix, a suitable buffer solution, a coloring enzyme, or a secondary antibody labeled with a fluorescent substance, a coloring substrate or the like for the immunological detection of antibody. As for the matrix, a nitrocellulose membrane, a 96 well plate made of polyvinyl resin, a 96 well plate made of polystyrene resin, and a sliding glass may be used. As for the coloring enzyme, peroxidase and alkaline phosphatase may be used. As for the fluorescent substance, fluorescein isothiocyanate (FITC) and rhodamine B isothiocyanate (RITC) may be used. As for the coloring substrate solution, ABTS(2,2′-azino-bis-(3-ethylbenzothiazoline-6-sulfonic acid)), OPD(o-phenylenediamine), or TMB(tetramethyl benzidine) may be used.

The present invention provides a method for providing information for the diagnosis of thyroid cancer, the method including:

(1) measuring the expression level of mRNA of the ADM2 gene or protein expressed from the gene from a biological sample isolated from a suspected thyroid cancer patient;

(2) comparing the expression level thereof with that of a normal control sample; and

(3) determining the thyroid cancer if the expression level thereof is over that of the normal control sample.

In the method for providing information for diagnosis of thyroid cancer of the present invention, the measurement of the expression level of mRNA or protein expressed from the gene from a biological sample may be performed by isolating mRNA of a gene or protein thereof from the biological sample, and the isolation of mRNA of the gene or protein thereof may be carried out using the method known in the art.

In the present invention, the term “biological sample” includes, but is not limited to, samples such as tissues, cells, whole blood, serum, plasma, tissue autopsy samples (brain, skin, lymph node, spinal cord, etc.), saliva, sputum, cerebrospinal fluid or urine. However, the biological sample may be used with or without manipulation.

In the method for providing information for diagnosis of thyroid cancer according to the present invention, when the measured expression level of mRNA of the ADM2 gene or protein expressed from the gene from a biological sample isolated from a suspected thyroid cancer patient is over that of the normal control sample, the suspected thyroid cancer patient can be diagnosed as a thyroid cancer patient.

The present invention provides a method for providing information for prognosis prediction of thyroid cancer, the method including:

(1) measuring the expression level of mRNA of the ADM2 gene or protein expressed from the gene from a biological sample isolated from an obese or recurrent thyroid cancer patient;

(2) comparing the expression level thereof with that of a normal body weight thyroid cancer patient sample; and

(3) determining that the risk of thyroid cancer exacerbation is high if the expression level thereof is over that of the normal body weight thyroid cancer patient sample.

The method for providing information for prognosis prediction of thyroid cancer of the present invention may measure the expression level of mRNA of the ADM2 gene or protein expressed from the gene from a biological sample isolated from an obese thyroid cancer patient and then compare the expression level thereof with that of a normal body weight thyroid cancer patient sample, thereby providing information on whether the prognosis of thyroid cancer patient is good or poor. Specifically, when the expression level of the ADM2 gene or protein of an obese thyroid cancer patient sample is over that of the normal body weight thyroid cancer patient sample, it is determined the risk of thyroid cancer exacerbation is high. Further, the method of the present invention may predict the risk of thyroid cancer exacerbation by identifying that the expression level of mRNA of the ADM2 gene or protein expressed from the gene from an obese and recurrent thyroid cancer patient sample is significantly increased compared with that from an obese thyroid cancer patient.

The analysis method for measuring the mRNA level and the analysis method for measuring the protein level are as described above. The comparison between the expression level of the marker gene or protein in normal body weight thyroid cancer patients and the expression level of the marker gene or protein in obese or recurrent thyroid cancer patients may be shown as absolute (e.g., μg/ml) or relative (e.g., the relative intensity of signal) differences.

The present invention provides a method of screening a therapeutic agent for thyroid cancer by measuring the expression level of ADM2 gene or protein encoded by the gene after administering the candidate substance expected to be capable of curing the thyroid cancer.

Specifically, the therapeutic agents may be screened by comparing a change (increase or decrease) of the ADM2 gene or the ADM2 protein under the presence or absence of a candidate substance for curing the thyroid cancer. Preferably an agent that indirectly or directly reduces the expression level of the ADM2 gene or the ADM2 protein can be selected as a therapeutic agent for thyroid cancer. In other words, the expression level of the ADM2 gene or ADM2 protein of the present invention in thyroid cancer cells may be measured in the absence of a candidate substance for curing thyroid cancer, and the expression level of the ADM2 gene or ADM2 protein of the present invention may be measured in the presence of a candidate substance for curing thyroid cancer so that the measured levels may be compared. The substance that reduces the expression level of the ADM2 gene or ADM2 protein of the present invention in the presence of a candidate substance for curing thyroid cancer compared that in the absence of a candidate substance for curing thyroid cancer may be predicted as a therapeutic agent for thyroid cancer.

Hereinafter, the present invention is described in more detail with reference to Examples. However, these Examples are for illustrative purposes only, and the present invention is not intended to be limited by these Examples.

Materials and Methods

Animal Experiments

Animals received humane care, and protocols were approved by the Institutional Animal Care and Use Committee of Chungnam National University School of Medicine (Daejeon, Korea). To generate thyrocyte-specific BRAF^{V600E} knock-in mice, lox-stop-lox (LSL)-Braf^{V600E} mice (a kind gift from James A Fagin, Memorial Sloan Kettering Cancer Center, N.Y., USA) were crossed with thyroglobulin-CreERT2 (TgCreER^{T2}) mice (a kind gift from Jukka Kero, University of Turku, Åbo FI-20521, Finland) on a C57BL/6 background [46,47]. Mice were acclimated to a 12 h light/12 h dark cycle, temperature (23° C.), and 50-60% humidity environment and fed a chow diet (CD). Tamoxifen (Sigma Aldrich, St. Louis, Mo., USA) was administered at a total dose of 5 mg for 5 days. The mouse model of DIO was developed by feeding HFD (60% of total energy intake as fat, TD. 06414, ENVIGO, Indianapolis, Ind., USA). Tissues were obtained when mice were euthanized. All mice used in this study were male.

RNA Sequencing

In the animal model of C57BL/6 mouse with thyroid carcinoma in which the LSL-BRAF mutation is specifically expressed in thyroglobulin, the thyroid tissue of a normal chow diet group or high fat diet (containing 10 times the fat content of normal chow diet) group and the thyroid tissue of control (normal animal model without LSL-BRAF mutation) were stored in Trizol. Then, RNA was extracted using Qiagen's RNA assay kit, and the BAM file was received from the Macrogen company for sequencing. Tuxedo protocol was used to select genes with significant differences in RNA sequencing. The analysis was performed twice: the first analysis was performed by dividing the genes into control and normal diet animal model with thyroid cancer, and second analysis was performed by dividing the genes into normal diet animal model with thyroid cancer and high-fat diet animal model with thyroid cancer.

Total RNA was isolated using TRIzol reagent (Life Technologies, Eugene, Oreg., USA) and transcribed into complementary DNA (cDNA) following the TruSeq Stranded Total RNA Sample Prep Guide (Illumina, San Diego, Calif., USA). Low-quality sequence artifacts were removed. Reads were mapped to the reference genome (Mus musculus, mm10) using HISAT2 or Kalisto. Differential expression analysis (DEA) was performed using the R package DEseq2. Transcript abundance was transformed into fragments per kilobase of transcript per million after transcript quantification. Gene set enrichment analysis (GSEA) was conducted with PIANO within the R program using gene sets obtained from Enrichr (https://maavanlab.cloud/Enrichr/).

Transmission Electron Microscopy

Thyroid tissues were fixed with 1% glutaraldehyde at 4° C. and washed with 0.1 M cacodylate buffer. Tissues were fixed for 1 h at 4° C. with 1% OsO₄ in 0.1 M cacodylate buffer, pH 7.2, containing 0.1% CaCl₂). They were then embedded in Embed-812 (Electron Microscopy Sciences, Hatfield, Pa., USA) and polymerized at 60° C. for 36 h. Embedded samples were sectioned using an EM UC6 ultramicrotome (Leica Biosystems, Heidelberger str, Nuβloch, Germany) and stained with 4% (w/v) uranyl acetate and lead citrate. Stained samples were examined using a Leo912 transmission electron microscope (Carl Zeiss, Oberkochen, Germamy) at 120 kV.

In Vitro Cell Culture

BCPAP and 8505c (DSMZ, Braunschweig, Germany) cells were cultured in RPMI 1640 medium (Welgene, Daejeon, Republic of Korea) supplemented with 10% fetal bovine serum (FBS; Gibco, Waltham, Mass., USA), 100 U/ml penicillin, and 100 μg/ml streptomycin at 37° C. in a humidified atmosphere with 5% CO₂. Rotenone (Sigma-Aldrich) was diluted with ethanol. Palmitate was conjugated with bovine serum albumin (BSA) according to the protocol of Seahorse Bioscience (North Billerica, Mass., USA). Cell lines were treated with 1 μM rotenone or 50 μM BSA-conjugated palmitic acid for 24 h. ADM2 recombinant protein (MyBioSource, San Diego, Calif., USA) was added to cell lines for 12 h. The genetic disruption of ADM2 was performed using human shRNA lentiviral particles (shADM2; TL306813V) from OriGene Technologies (Rockville, Md., USA). Scrambled shRNA lentiviral particles (shCtrl; TR30021V; OriGene Technologies) were used as controls.

Oxygen Consumption Rate Analysis

Cells were plated at a concentration of 1×10⁴ per well on Seahorse XF-24 plates (Seahorse Bioscience) overnight. BSA-conjugated palmitic acid was added for 24 h after the cells had attached to the wells. A calibration cartridge (#102416-100, Seahorse Bioscience) was activated overnight in a non-CO₂-containing incubator. On the measurement day, the cell medium was changed to XF assay medium (#102365-100, Seahorse Bioscience), and cells were incubated in a non-CO₂-containing incubator for 1 h. The XF-24 analyser was calibrated using the calibration cartridge. After calibration, the cell plate was loaded onto Seahorse XF-24 extracellular flux analyser (Seahorse Bioscience), and mitochondrial function was measured with inhibitors: oligomycin (2 μg/ml), carbonyl cyanide 3-chlorophenylhydrazone (10 μM), and rotenone (1 μM).

Analysis of the Genotype-Tissue Expression (GTEx) and the Cancer Genome Atlas (TCGA) Data Set

Survival analyses were performed using the GEPIA2 (http://gepia2.cancer-pku.cn/). Expression levels of ADM2 and RAMPs were downloaded from the GTEx portal (http://gtexportal.org/). The TCGA-THCA dataset was obtained from the UCSC database (http://xena.ucse.edu/). To elucidate the role of ADM2, we divided THCA into two groups based on ADM2 expression by quartile. DEA and GSEA were performed using DESeq2 and PIANO.

Statistical Analyses

The data shown represent at least three independent experiments. The in vivo data were replicated using individual mice. Data are expressed as mean±standard error (SEM). Statistical analyses were performed using GraphPad Prism 9 (GraphPad Software, San Diego, Calif., USA). Data were analysed using one-way analysis of variance (more than two groups), Student's two-tailed t-test (two groups), or univariate analysis using stepwise logistic regression.

Sample Preparation

Sample Preparation of Animal Model with Thyroid Cancer

LSL-Braf^{V600E} X TPO-Cre mice previously used in other studies is an animal model in which mutations of LSL-Braf^{V600E} is induced when TPO-Cre is expressed at 14.5 days of the embryonic day, resulting in the induction of thyroid cancer from the developmental stage of mouse thyroid. Thus, it can be inconsistent with the timing of human thyroid cancer. The human thyroid cancer occurs in the form of a thyroid nodule in the normal thyroid parenchyma constituting normal thyroid follicular structure, whereas the existing thyroid-specific carcinoma animal model using TPO-Cre is a maligned model because the follicular structure is destroyed in the entire thyroid so that it is not suitable to reflect the physiology of the patient.

The thyroid cancer animal model used in the present invention was a model in which Tg-Cre/ERT2 mouse (provided by Prof Jukka Kero of the University of Turku, Finland, genesis, 2014, 52, 333 to 340) and LSL-Braf^{V600E} mouse (provided by Prof. Shioko Kimura of the National Institutes of Health, PNAS, 2011, 108(4), 1615 to 1620) were cross-bred and they were injected with Tamoxifen to express Tg, thereby inducing the mutation of Braf^{V600E}. In order to evaluate whether the 8-week-old thyroid cancer model, which can be considered to have normal thyroid development, was suitable as a carcinoma animal model after adult development, sixteen males were treated with LSL-Braf^{V600E} X Tg-Cre/ERT2 with Tamoxifen for one week to induce mutation of Tg-specific Braf^{V600E} to produce sixteen animal models with thyroid cancer. Eight 8-week-old males were injected with vehicle (saline) to prepare the control group. From the 10th week to the 22nd week, the control and eight thyroid cancer animal models were fed normal diet, and the remaining eight thyroid cancer animal models were fed high fat diet (Casein 265 g/kg, L-Cystine 4 g/kg, Maltodextrin 160 g/kg, Sucrose 90 g/kg, Lard 310 g/kg, Soybean Oil 30 g/kg, Cellulose 65.5 g/kg, Mineral Mix 48 g/kg, Calcium Phosphate 3.4 g/kg, Vitamin Mix 21 g/kg, Choline Bitartrate 3 g/kg and Blue Food Color 0.1 g/kg; ENVIGO, TD. 06414) for about 12 weeks. At 22nd weeks of age, mice were sacrificed, and morphological changes of the thyroid gland were observed by immunohistochemical staining (IHC). Histological characteristic was analyzed on eight individuals, and RNA sequencing was performed on nine individuals, including three individuals per group.

Sample Preparation of Patient with Thyroid Cancer

In order to develop a gene marker for diagnosis and prognosis prediction of thyroid cancer, subjects were collected, and the expression levels of ADM2 thereof were analyzed using their thyroid cancer tissue samples. Of the subjects who were collected, the experiment group was selected based on the criteria shown in Table 1 below. A total of 30 normal body weight thyroid cancer patients, 40 obese thyroid cancer patients, and 10 obese patients with recurrent thyroid cancer were finally selected.

TABLE 1
Thyroid cancer patient classification

| | |
|---|---|
| Normal body weight patient group | Person who has no hypertension, diabetes, hyperlipidemia, |
| among thyroid cancer patients | heart disease, stroke (cognitive, non-cognitive). |
| | Person who does not take blood coagulant. |
| | Person with a BMI of 19 or more and 25 or less. |
| Obese patient group among thyroid | Person who has no hypertension, diabetes, hyperlipidemia, |
| cancer patients | heart disease, stroke (cognitive, non-cognitive). |
| | Person who does not take blood coagulant. |
| | Person with a BMI of 25 or more. |
| Recurrent patient group among | Person who has no hypertension, diabetes, hyperlipidemia, |
| obese thyroid cancer patients | heart disease, stroke (cognitive, non-cognitive). |
| | Person who does not take blood coagulant. |
| | Person with a BMI of 25 or more. |
| | Person who was evaluated to have recurrence by the |
| | ultrasonography of the thyroid for evaluating the recurrence |
| | of thyroid cancer |
| | and who was diagnosed as recurrence of imaging and |
| | biochemistry by confirming the increase of serum |
| | thyroglobulin |

The present invention was conducted with the approval of the Institutional Review Board of Chungnam National University. Clinical questionnaire data collection and human body collection were conducted after obtaining the written consent of the subjects.

Immunohistochemical Staining (THC)

Slides with H & E staining (Hematoxylin and eosin stain) for all cases with good storage and good fixation among 80 cases of patients diagnosed as thyroid cancer were reviewed. Tissues were fixed in formalin, and then the tissues embedded in paraffin were microtome-sliced to 5 μm thickness. The sliced tissues were fixed on slides. They were incubated in a constant temperature oven at 60° C. for 1 hour. Then, they were deparaffinized in a conventional manner, and then treated with different concentrations of alcohol and washed with distilled water. In order to inhibit the activity of endogenous peroxidase, they were treated with 3% aqueous hydrogen peroxide (H₂O₂) for 20 minutes. Then, they were washed with Tris buffer solution (Tris 3.025 mg, 1 M NaCl 40 g, 1 M HCl 22 ml in H₂O 5 L, pH 7.4). For antigen recovery, the pressure cooker was filled with citrate buffer solution (sodium citrate 14.7 g, 1 M HCl 27 ml in H₂O 5 L, pH 6.0) and was heated. The slides were placed at the start of the boiling, and after the pressure reached the maximum (130 kPa), it was boiled for 2 more minutes, and immersed in cold water to lower the pressure. The slides were taken out and placed in Tris buffer solution. To prevent nonspecific reactions, the blocking antibody was reacted at room temperature for 10 minutes, and the primary antibody was reacted. Anti-adrenomedullin 2 antibody (SC-140883, Santa Cruz Biotechnology, USA) was used as the primary antibody. After the primary antibody reaction, they were washed with Tris buffer solution, and LSAB+ kit (DAKO, Japan) was used to react with the secondary antibody at room temperature for 30 minutes. After completion of the reaction, they were washed with water to complete the color reaction. Then, they were stained with Harris hematoxylin, dehydrated with alcohol, sealed, and observed with a microscope.

### Example 1. Analysis of Role of ADM2 in Animal Model with Thyroid Cancer

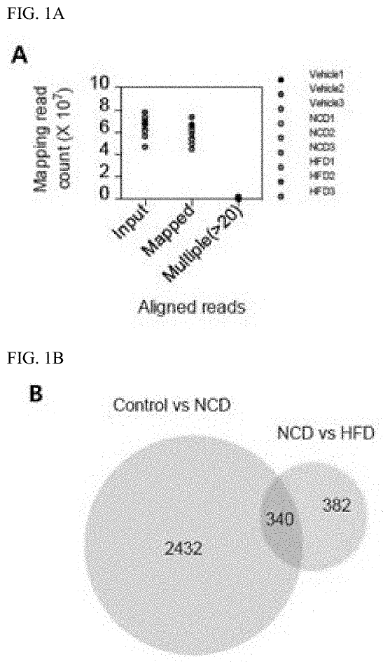

In order to analyze the role of ADM2 in animal models with thyroid cancer, expression levels of ADM2 in animal model tissues were identified. As a result, it was observed that the high-fat diet group (HFD) showed the increase in the size of thyroid cancer and the number of proliferating cells compared with the normal chow diet (NCD) in the animal model with the thyroid cancer. In order to identify the mechanism, RNA sequencing was performed. As a result, about 2,732 genes were differentiated in the cancer-free control and NCD animal models, and about 722 genes were statistically differentiated in the NCD animal model and the HFD animal model. Among these, ADM2 (adrenomedullin 2) gene was found to be a gene which differs in all of the control, NCD and HFD (See FIG. 1).

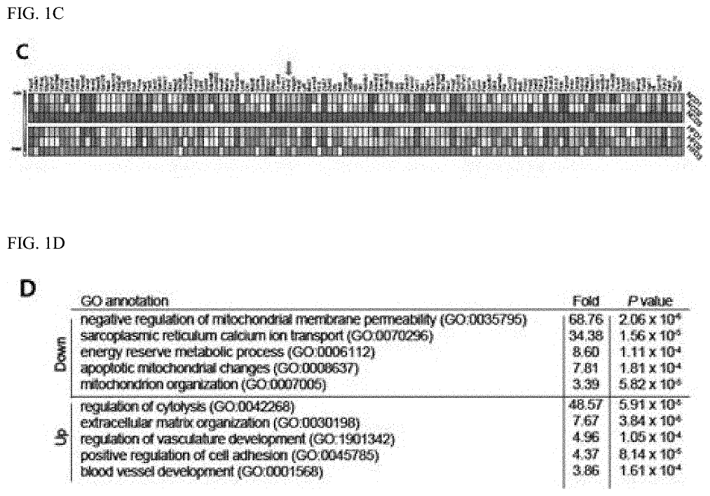

ADM2 protein expression in the animal model with thyroid cancer was confirmed, and the results indicated that the expression of ADM2 protein in the HFD animal model was significantly increased as compared with the NCD animal model (See FIG. 2).

### Example 2. Analysis of Role of ADM2 in Patient with Thyroid Cancer

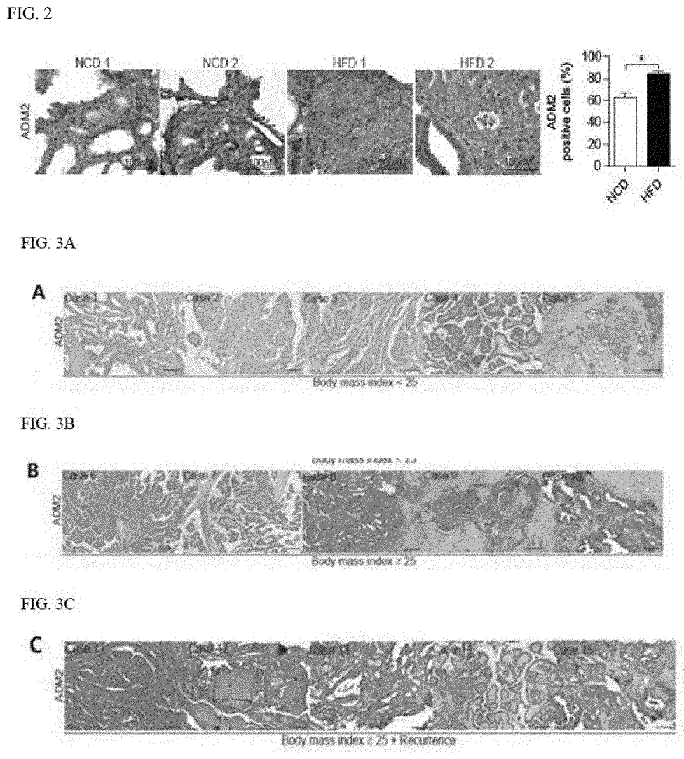

In order to analyze the role of ADM2 in patients with thyroid cancer, the expression level of ADM2 of thyroid cancer patient sample was identified. The results indicated that compared to those of normal thyroid cancer (FIG. 3A), the ADM2 expression was increased in tissues of thyroid cancer (FIG. 3B) of obese patients with thyroid cancer, whose clinical features such as lymph node metastasis and recurrence of thyroid cancer were worse, and the ADM2 expression was further increased in tissues of thyroid cancer (FIG. 3C) of obese patients with recurrent thyroid cancer compared to those of obese patients with thyroid cancer.

The above results show that the expression level of ADM2 gene can be used as a therapeutic composition for solving intractable thyroid cancer because it can predict the risk of recurrence after treatment of thyroid cancer as well as the relationship between obesity and exacerbation of thyroid cancer.

As described above, the preferred exemplary embodiments have been described, but the scope of the present invention is not limited thereto. Various modifications and improvements by those skilled in the art using the basic concept of the present invention defined in the following claims are also within the scope of the present invention.

As described above, the exemplary embodiments have been described and illustrated in the drawings and the specification. The exemplary embodiments were chosen and described in order to explain certain principles of the invention and their practical application, to thereby enable others skilled in the art to make and utilize various exemplary embodiments of the present invention, as well as various alternatives and modifications thereof. As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

Characteristics of the Thyrocyte-Specific Braf^{V600E}-Inducible Mice

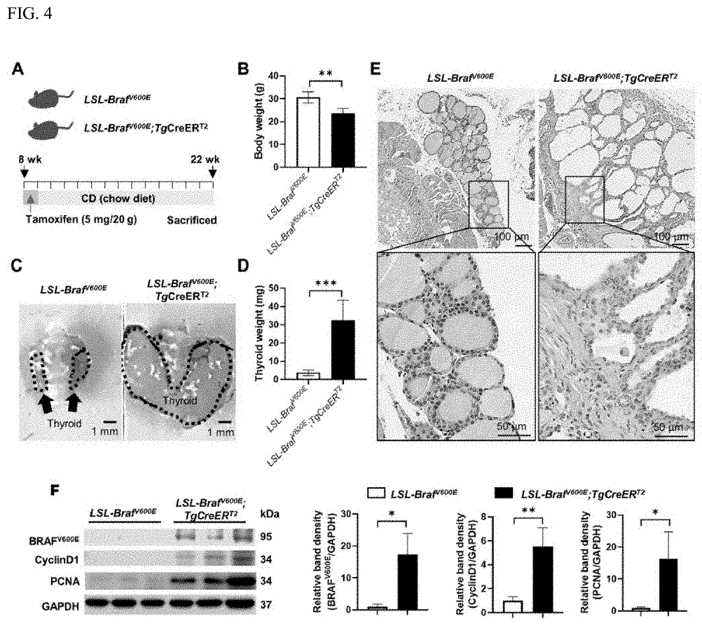

The present invention discloses a mouse model for inducing thyrocyte-specific BRAF^{V600E} mutations by crossing LSL-Braf⁶⁰⁰E and TgCreER² mice to generate LSL-BRAF^{V600E}; TgCreER^{T2} mice. Tamoxifen was administered at 8 weeks of age to induce expression of BRAF^{V600E}, and the mice were sacrificed at 22 weeks of age (FIG. 4A). The body weight of LSL-BRAF^{V600E}; TgCreER^{T2} mice was lower than that of the LSL-BRAF^{V600E} mice (FIG. 4B). LSL-BRAF^{V600E}; TgCreER^{T2} mice developed goitres (FIG. 4C). LSL-BRAF^{V600E}; TgCreER^{T2} mice had significantly increased thyroid weight compared to LSL-BRAF^{V600E} mice (FIG. 4D). The thyroids of LSL-BRAF^{V600E}; TgCreER^{T2} mice had dysmorphic and variable follicles and formed a solid mass region with papillary growth (FIG. 4E). The levels of BRAF^{V600E} and proliferation-related proteins (CyclinD1 and PCNA) were significantly increased in LSL-BRAF^{V600E}; TgCreER² mice compared to LSL-BRAF^{V600E} mice (FIG. 4F).

Impact of a High-Fat Diet on Thyrocyte-Specific BrafV600E-Inducible Mice

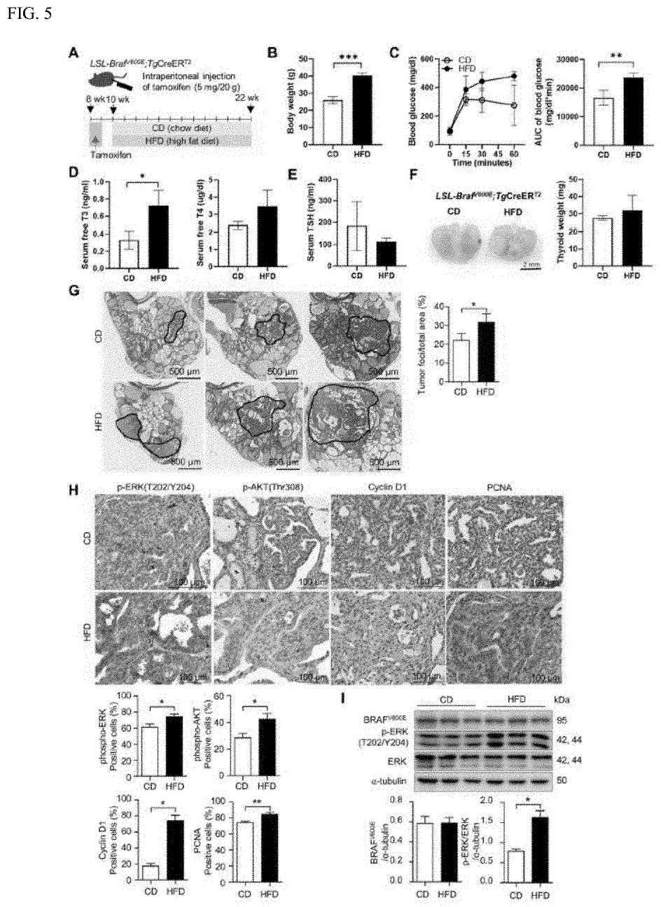

To investigate the effect of obesity on thyroid cancer behaviour, LSL-BRAF^{V600E}; TgCreER^{T2} mice were put on HFD after tamoxifen administration (FIG. 5A). LSL-BRAF^{V600E}; TgCreER^{T2} mice fed HFD for 12 weeks showed increased body weight, white adipose tissue, and liver weight compared to CD-fed mice (FIG. 5B) and developed systemic glucose tolerance (FIG. 5C). Moreover, plasma levels of ALT and total cholesterol increased in H1FD-fed mice. Serum free T3 increased in HFD-fed compared with that in CD-fed mice. There was no change in serum free T4 and thyroid-stimulating hormone (TSH) concentration (FIG. 5D, E). H1FD-fed mice showed an increased percentage of tumour foci/total thyroid area, although there was no difference in thyroid weight (FIG. 5F, G). Furthermore, HFD-fed mice had an increased proportion of nuclear clearing cells and oncocytic-changed cells, which is related to the accumulation of abnormal mitochondria. Increased phospho-ERK (T202/Y204), phospho-AKT (Thr308), Cyclin D1, and PCNA levels were observed in H1FD-fed mice (FIG. 5H); however, the expression of BRAF^{V600E} was similar in the two groups (FIG. 5I). Additionally, western blotting analysis of the entire thyroid gland including non-tumour foci, showed increased p-AKT and an increasing trend for PCNA and CyclinD1 in H1FD-fed mice. The expression of immune cell markers (CD4, CD8, and CD163) was not detected in the tumour region in LSL-BRAF^{V600E}; TgCreER^{T2} mice fed with either CD or HFD. Lymphovascular invasion and extrathyroid extension were absent in both groups (data not shown). These results show that HFD activated thyroid tumour proliferation through additional factors via the ERK signalling pathway.

Induction of ADM2 by Metabolic Stress

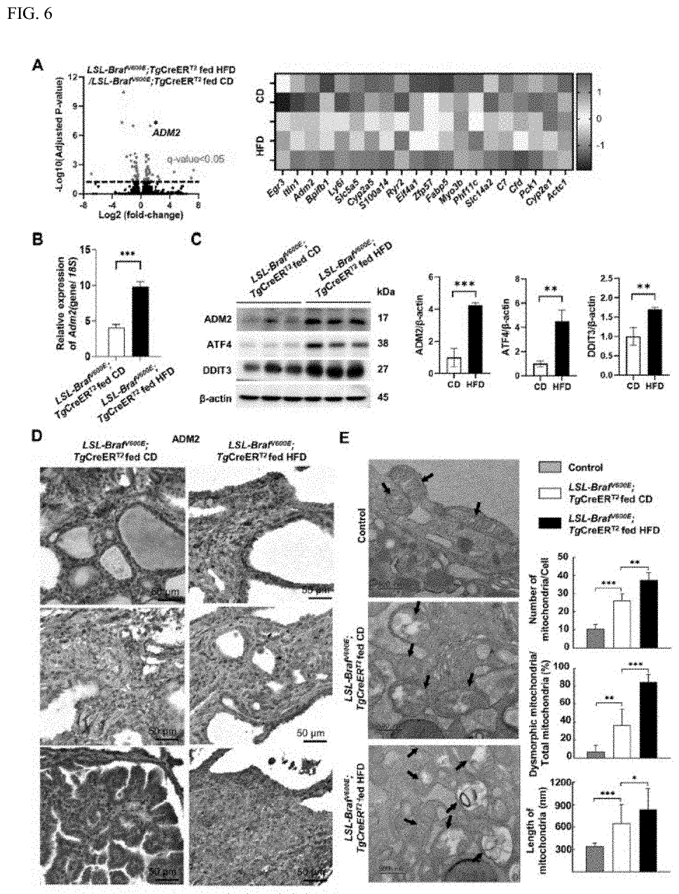

To identify the factors related to the progressive features of thyroid tumours in HFD-fed LSL-Braf^{V600E}; TgCreER^{T2} mice, we analysed transcriptomes. Among the 20 genes that showed significantly altered expression (adjusted p-value<0.05), ADM2 was the most significantly increased gene in mice on HFD compared to CD (FIG. 6A). The expression of ADM2 was upregulated approximately 2-fold in LSL-Braf^{V600E}; TgCreER^{T2} mice fed HFD compared to those fed CD according to RT-qPCR (FIG. 6B). Additionally, HFD-fed mice had increased expressions of ADM2 and ISR-related proteins (ATF4 and DDIT3) (FIG. 6C). Immunohistochemistry showed that although tumour cells expressed ADM2 in LSL-Braf^{V600E}; TgCreER^{T2} mice fed CD, HFD-fed mice had a larger population of ADM2-positive cells and increased expression of ADM2 in tumour cells (FIG. 6D). HFD-fed LSL-Braf^{V600E} mice exhibited increased ADM2 and reduced phospho-ERK expression compared to CD-fed LSL-Braf^{V600E} mice, although no differences were observed in thyroid size between groups. We observed increased cells with an oncocytic change in LSL-Braf^{V600E}; TgCreER^{T2} mice fed an HFD. In addition, we found that thyrocytes of HFD-fed mice had dysmorphic mitochondria, which were swollen and had collapsed cristae and an increased number of mitochondria than CD-fed mice (FIG. 6E). Collectively, our findings indicate that HFD induced ADM2 expression in thyrocytes with dysmorphic mitochondria in LSL-Braf^{V600E}; TgCreER^{T2} mice.

Upregulation of ADM2 by Mitochondrial Complex I Inhibitor or Palmitic Acid Treatment

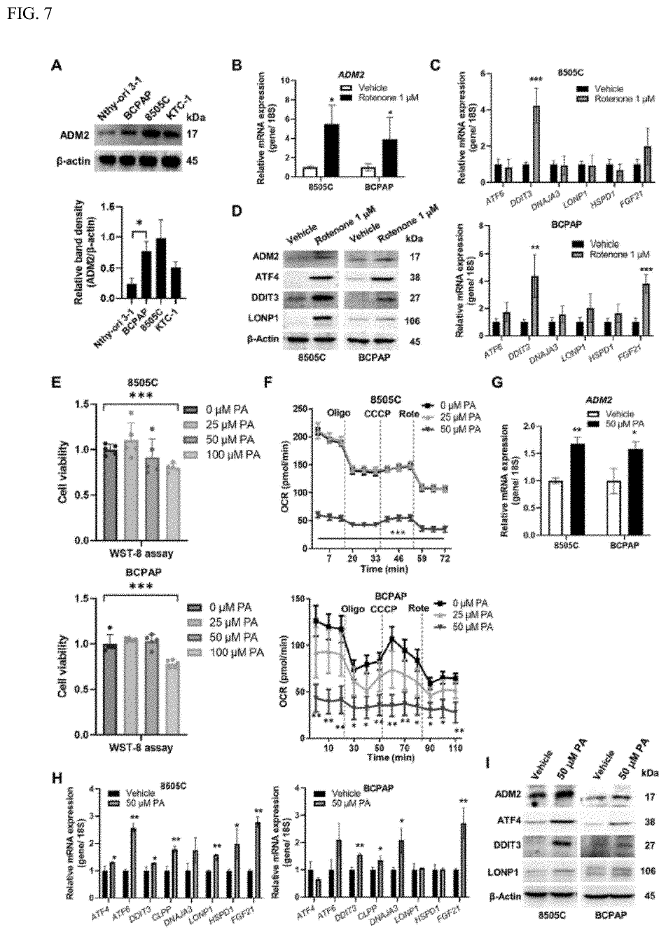

Compared with the immortalized thyroid cell line (Nthy-ori 3-1), the BRAF mutated cancer cell lines (BCPAP, 8505 C, KTC-1) had increased expression of ADM2 (FIG. 7A). The expression of ADM2 was analysed in BCPAP, a papillary thyroid cancer (PTC) cell line, and 8505 C, an anaplastic thyroid cancer cell line, which originated from a primary region of cancer. We examined whether the expression of ADM2 was regulated by metabolic stress, particularly the mitochondrial stress response. Treatment with rotenone, an inhibitor of OxPhos complex I, led to a significant increase in ADM2 expression in 8505 C and BCPAP cells (FIG. 7B). In addition, rotenone induced increased expression of the ISR-related genes DDIT3 in both cancer lines and FGF21, in BCPAP cells (FIG. 7C), and protein expressions of ADM2, ATF4, DDIT3, and LONP1 were upregulated by rotenone (FIG. 7D).

Next, we examined mitochondrial function and ADM2 expression after fatty acid treatment. Palmitic acid, the most common saturated fatty acid, is increased in obesity and induces intracellular metabolic stress. We performed an oxygen consumption rate analysis to measure mitochondrial function in thyroid cancer cells treated with palmitic acid. There was no difference in viability up to 50 μM palmitic acid, but viability decreased at 100 μM (FIG. 7E). Cancer cell lines treated with 50 μM palmitic acid reduced their oxygen respiration rates compared with vehicle controls (FIG. 7F). Palmitic acid stimulated the expression of ADM2 in thyroid cancer cell lines (FIG. 7G) and induced the expression of ATF4, ATF6, DDIT3, CLPP, LONP1, HSPD1, and FGF21 in 8505 C cells and DDIT3, CLPP, DNAJA3, and FGF21 in BCPAP cells (FIG. 7H). The expression of the proteins ADM2, ATF4, DDIT3, and LONP1 also increased (FIG. 7I). Collectively, these results show that metabolic stress stimulates ADM2 expression along with induction of the ISR in thyroid cancer cell lines.

Cell Proliferation and Migration by ADM2 Via ERK Signalling In Vitro.

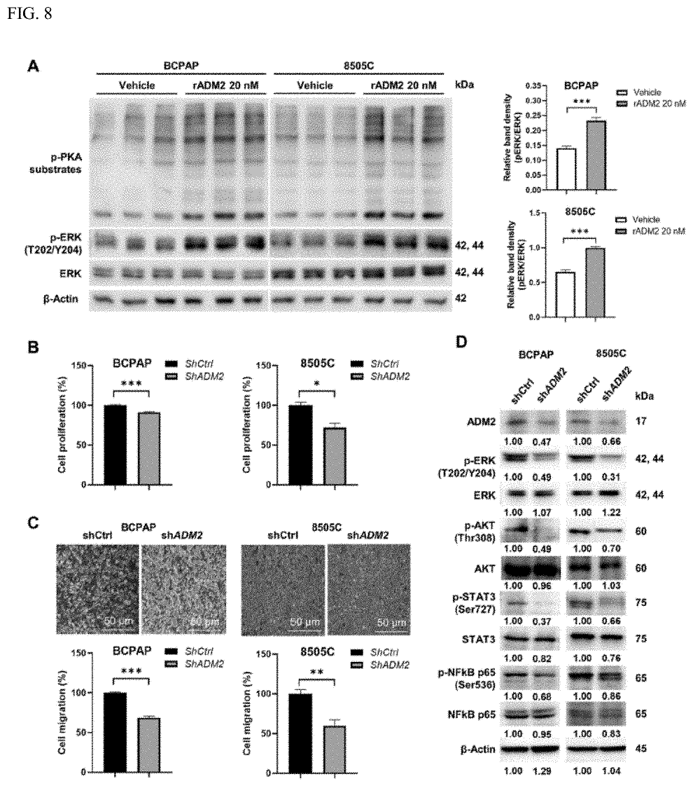

To ascertain whether ADM2 accelerates cancer progression, we analysed the effects of recombinant ADM2 treatment or shADM2 in a cancer cell line. First, we confirmed that ERK was phosphorylated by recombinant ADM2. Treating cells with 20 nM of recombinant ADM2 resulted in the phosphorylation of PKA substrates and ERK (T202/Y204) in the thyroid cancer cell lines (FIG. 8A). Knockdown of ADM2 was performed. Transfection with shAdm2 reduced the expression of ADM2 in cancer cell lines (FIG. 8D). The shADM2-transfected cell lines showed reduced proliferation and migration than the shCtrl-transfected cell line (FIG. 8B, C). Moreover, shADM2-transfected cells showed decreased phosphorylation of ERK (T202/Y204), AKT (Thr308), STAT3 (Ser727), and NF-KB (Ser536) compared to shCtrl-transfected cells (FIG. 8D). These results demonstrate that ADM2 promotes thyroid cancer progression by stimulating the phosphorylation of ERK signalling.

Association of ADM2 with the ISR Pathway and Poor Survival Rates in Thyroid Cancer in Humans

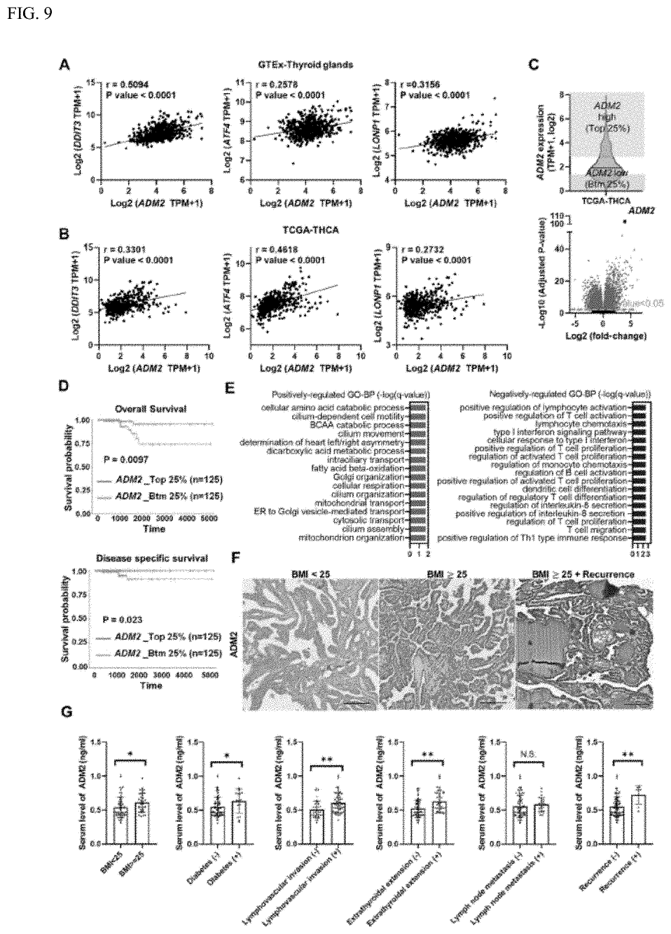

To elucidate the role of ADM2 in human subjects, we investigated RNAseq data from the human databases GTEx (a public database providing non-disease tissue for molecular assays, including RNAseq) and TCGA (a landmark genomics program for cancer providing molecular characteristics). We compared ADM2 expression between organs. The expression of ADM2 in the thyroid gland was 19.1, while the mean expression of ADM2, excluding the thyroid gland, was 1.15. The kidney had the second-highest expression value, which was 5.8. Using correlation analysis, we examined whether the expression of ADM2 was associated with metabolic stress genes. Expression of ISR genes (DDIT3, ATF4, and LONP1) was significantly correlated with ADM2 expression in the normal thyroid gland (FIG. 9A) and the thyroid cancer dataset (FIG. 6B). However, expression of ADM2 and ATF4 was reduced in tumour tissues, although LONP1 expression increased. These data show the distinct role of ADM2 and ISR in normal and tumour tissues. To predict the role of ADM2 in tumour aggressiveness, patients with thyroid cancer were classified according to ADM2 expression levels into a top (high ADM2 expression) and bottom 25% group (low ADM2 expression) (FIG. 9C). Interestingly, the top 25% group showed lower overall and disease-specific survival than that of the bottom 25% group (logrank p=0.043) (FIG. 9D). The same trend was found in the Non-BRAF and BRAF subtypes, although it was not significant. There was no difference in survival between the groups classified by ADM2 receptors (RAMP1, RAMP2, and RAMP3). We identified the pathways related to ADM2 using gene ontology analysis. Interestingly, fatty acid beta-oxidation, mitochondrial transport, and mitochondrial organization were upregulated in the top 25% group. Furthermore, we observed that differentially expressed genes were associated with fatty acid degradation, the tricarboxylic acid cycle, and oxidative phosphorylation pathways in the Kyoto Encyclopedia of Genes and Genomes (KEGG) (FIG. 9E). By contrast, immunerelated processes were enriched in the group with low ADM2 expression (FIG. 9E). Collectively, these results show that ADM2 is potentially an oncogenic factor in silico as well as in vivo and in vitro.

Association of ADM2 with Aggressive Clinicopathologic Parameters and Obesity in Thyroid Cancer

To evaluate the relationship between obesity-induced ADM2 expression and tumour aggressiveness, we investigated the clinicopathologic features, including body mass index, associated with samples from 160 PTC patients treated at Chungnam National University Hospital (CNUH) from 2003 to 2010. Patients were divided into two groups according to ADM2 immunoreactivity. High ADM2 expression was significantly associated with clinicopathological parameters, including body mass index, tumour size, lymph node metastasis, and locoregional recurrence. To assess the usefulness of ADM2 expression as an independent predictor of aggressive PTC phenotypes, multivariate analysis using stepwise logistic regression was conducted (Tables 2 and 3). This showed that high ADM2 expression was an independent risk factor for body mass index (p=0.003, odds ratio [OR] 1.236), tumour size (p=0.030, odds ratio [OR] 1.802), and the presence of lymph node (LN) metastasis (p=0.019, [OR] 2.330). Thus, ADM2 expression was highly associated with obesity and tumour recurrence (FIG. 9F).

TABLE 2
Logistic regression analysis of the relationship between
ADM2 staining and clinicopathological factors.

| Factors | Exp(β) | SE | 95.0% CI | p-value |
|---|---|---|---|---|
| Body mass index | 1.236 | 0.072 | (1.074. 1.424) | 0.003* |
| Tumour size | 1.802 | 0.272 | (1.058, 3.069) | 0.030* |
| Lymph node metastasis | 2.330 | 0.656 | (1.147. 4.733) | 0.019* |
| Recurrence | 1.685 | 0.656 | (0.466, 6.095) | 0.173 |

SE, standard error; Exp(β), odds ratio; CI, confidence interval, *statistically significant

TABLE 3
Variables and No. of patients with ADM2 levels.

| | | | ADM2 | | |
|---|---|---|---|---|---|
| | | No. of | Low | High | |
| Variables | | patients | (n = 72) | (n = 88) | p-value |
| Age, years | | 160 | 47.1 ± 12.5 | 48.4 ± 13.0 | 0.512 |
| Body mass index | | 160 | 23.2 ± 2.9 | 24.7 ± 2.5 | 0.002* |
| (BMI) | | | | | |
| Gender | Male | 27 | 9 | 18 | 0.181 |
| | Female | 133 | 63 | 70 | |
| Tumor size | | 160 | 1.13 ± 0.60 | 1.52 ± 0.93 | 0.003* |
| Multicentricity | No | 91 | 35 | 56 | 0.056 |
| | Yes | 69 | 37 | 32 | |
| Microscopic | No | 40 | 21 | 19 | 0.271 |
| capsular invasion | Yes | 120 | 51 | 69 | |
| Extrathryoid | No | 123 | 58 | 65 | 0.318 |
| extension | Yes | 37 | 14 | 23 | |
| Lymphovascular | No | 38 | 18 | 20 | 0.737 |
| invasion | Yes | 122 | 54 | 68 | |
| Central lymph | No | 112 | 57 | 55 | 0.022* |
| nodemetastasis | Yes | 48 | 15 | 33 | |
| Lateral lymph | No | 152 | 71 | 81 | 0.058 |
| node metastasis | Yes | 8 | 1 | 7 | |
| Locoregional | No | 142 | 68 | 74 | 0.039* |
| recurrence | Yes | 18 | 4 | 14 | |
| BRAF^{V600E} | No | 29 | 17 | 12 | 0.103 |
| mutation | Yes | 131 | 55 | 76 | |

Patients were divided into two groups according to their ADM2 serum levels. The high circulating ADM2 group showed increased body mass index and fasting glucose levels. Moreover, circulating ADM2 levels were significantly associated with the presence of extrathyroidal extension, lymphovascular invasion, and lymph node metastasis. The ADM2 levels were significantly higher in obese patients (BMI>25) than in lean patients (BMI<25). Patients with diabetes showed increased ADM2 levels compared to non-diabetic patients. Furthermore, the mean ADM2 level was higher in the patients with lymphovascular invasion and extrathyroidal extension than in patients without these conditions (FIG. 9G). Interestingly, patients in the recurrent group had significantly higher ADM2 serum levels than those in the nonrecurrent group. The results of the human data indicate that ADM2 is strongly associated with obesity and tumour aggressiveness.

The present invention attempted to identify the cell-autonomous, and non-cell-autonomous factors and pathways that determine thyroid cancer progression using thyrocyte-specific BRAFV600E-inducible mice fed an HFD. Thyroid tumour cells in thyrocyte-specific BRAFV600E-inducible mice fed HFD showed dysmorphic follicular changes with mitochondrial ultrastructural alterations. ADM2 is a prominent secretory factor in tumour cells of HFD-fed thyrocyte-specific BRAFV600E-inducible mice. Upregulation of ADM2 in tumour cells was enhanced by activation of the mitochondrial stress response pathway following fatty acid treatment in vitro. Furthermore, ADM2 expression is augmented in obese patients with thyroid cancer who show larger tumour size, high prevalence of lymph node metastasis, and locoregional recurrence.

ThrbPVPVPten^{+/-} mice with thyroid cancer developed by knock-in dominant-negative mutation of the thyroid hormone receptor R (Thrb) gene and deletion of one allele of the Pten gene harbour larger tumours with anaplastic phenotypes after being given an HFD. Increased activation of JAK-STAT3 signalling pathways that may be activated by leptin, which originates from adipocytes in expanded fat pads with a high-fat diet. This (ThrbPVPVPten^{+/-}) is a representative example of an adipose tissue-adipokine-thyroid tumour connection in an animal model of thyroid cancer, which has the characteristic features of follicular thyroid cancer. In the present invention, we did not validate the role of leptin-JAK-STAT3 activation in enhancing the expression of ADM2. However, evidence strongly shows that thyroid tumour behaviour can be transformed into more progressive phenotypes with excessive high fat nutrition.

In the rat thyroid follicular cell line FRTL-5, ADM2 increased by TSH; however, in the present model, serum TSH levels remained unaltered by overnutrition compared with that in control mice (FIG. 5E), showing that there were other factors inducing ADM2. Since we had previously identified the induction of mitokines by the ISR pathway and as our electron microscopy data showed increased dysmorphic mitochondria, we hypothesized that ADM2 expression increased by ISR pathways. The transcription factor ATF4, which integrates cellular stresses, has been considered a major factor in stress-inducible ADM2 expression by regulating the ADM2 promoter. Interestingly, secreted ADM2 plays a protective role in endoplasmic reticulum stress-induced myocardial injury through the PI3K-AKT signalling pathway. These findings shows that ADM2 may play a hormetic role in overcoming cellular stress under physiological and pathological conditions. Consistent with previous studies, we demonstrated that ADM2 expression increased with the activation of ATFs and DDIT3 in response to metabolic stresses, inducing mitochondrial inhibition (palmitic acid and rotenone), in thyroid cancer. Elevated levels of ADM2 in thyroid cancer may reduce the stress response; however, ADM2 plays a role in cancer progression.

In the TCGA database, ADM2 was related to worse survival in total tumour and subtypes in relation to the presence of BRAFV600E, although ADM2 decreased in tumour tissues compared with normal thyroid. These results indicate the tumour-specific role of ADM2 in regulating tumourigenesis independent of the BRAF mutation in thyroid cancer.

ADM2 was a predictor of survival in pancreatic cancer, and preoperative plasma ADM2 levels were identified as independent predictors of 5-year mortality, disease-free survival, and overall survival. In addition, blocking ADM2 regulates ERK and Gli1-Bcl2 signalling, which are important for cell proliferation and survival in hepatocellular carcinoma. Notably, increased ADM2 and decreased p-ERK levels were observed in healthy (non-cancerous) thyroid glands in the HFD group. It is unclear whether ADM2 is directly involved in ERK phosphorylation in healthy thyroid glands; this might be a physiological difference due to the presence or absence of an oncogene. Nevertheless, we identified that ADM2 stimulates proliferation and migration by enhancing ERK signalling and is associated with aggressive clinicopathological factors in thyroid cancer.

As mentioned above, plasma and cellular levels of ADM2 are valuable prognostic markers in patients with cancer. However, the role of ADM2 in obese cancer patients has not yet been elucidated, and changes in the plasma and cellular levels of ADM2 have not yet been examined. The present invention showed that ADM2 was increased in the thyroid of HFD-fed mice in both cancer and noncancer models (FIG. 6). Additionally, our results showed that blood ADM2 levels were increased when thyroid cancer was present with obesity as a comorbidity (FIG. 6G). Interestingly, whilst circulating ADM2 is inversely correlated with obesity in humans, ADM2 levels in adipose tissue are increased during obesity. These findings demonstrate that circulating ADM2 originates from various tissues, including the thyroid gland, and is differentially regulated between physiological and pathological (cancerous) conditions. Therefore, the present invention shows that ADM2 may play a specific role as a biomarker for predicting the progression of thyroid cancer in obese patients.

The present invention discloses a role for ADM2 in cancer cells. Although immune cell markers were not detected in the tumour of the mouse model, we found an association between ADM2 and inflammatory response in TCGA-THCA data.

ADM2 reduces organ injury (lung and kidney) and mortality by repairing vascular leakage and alleviating inflammatory responses, and alleviation of inflammatory response is accompanied by reduced macrophage infiltration. In contrast to the septic condition, a reduced inflammatory response can be beneficial for tumour survival. In previous studies, we elucidated the roles of FGF21 and GDF15 as mitokines in thyroid cancer. We found that serum FGF21 levels were associated with recurrence and body mass index. Recombinant FGF21 induces epithelial-to-mesenchymal transition signalling in cancer cells and increases the migration of thyroid cancer cell lines. Although Fgf21-related genes were not observed in BRAF-mutant mice fed HFD (FIG. 6A), there is a possibility that increased serum FGF21 in obesity contributes to the progression of thyroid cancer. Interestingly, although ADM2 and GDF15 have common upstream regulators (ATFs and CHOP), the correlation result of TCGA-THCA shows that ADM2 and GDF15 were negatively correlated. These results show that although ADM2 and GDF15 can be regulated as mitokines, their detailed roles and induction conditions may differ.

The present invention shows the role of ADM2 in the nexus of obesity and thyroid cancer. It shows that ADM2 may be used as a therapeutic target in thyroid cancer and a useful
biomarker for predicting thyroid tumour progression.

1. A composition for diagnosis or prognosis prediction of thyroid cancer, the composition comprising an agent for measuring an expression level of mRNA of ADM2 (adrenomedullin 2) gene or its protein.

2. The composition of claim 1, wherein the agent for measuring the level of mRNA of the gene includes a primer, a probe, or an antisense oligonucleotide that specifically binds to the gene.

3. The composition of claim 1, wherein the agent for measuring the level of the protein includes an antibody or an aptamer specific for the protein.

4. A method for providing information for a prognosis of a thyroid cancer patient, the method comprising:
a) measuring an expression level of ADM2 protein expressed from ADM2 gene in a biological sample isolated from an obese patient with thyroid cancer or recurrent thyroid cancer patient,
wherein
the expression level is measured by a method selected from the group consisting of western blot, enzyme-linked immunosorbent assay, radioimmunoassay, adioimmunodiffusion, Ouchterlony immunodiffusion, rocket immunoelectrophoresis, immunohistochemistry, immunoprecipitation assay, complement fixation assay, fluorescence activated cell sorting, and protein chips,
the biological sample is one selected from the group consisting of tissues, cells, whole blood, serum, plasma, tissue autopsy samples, saliva, sputum, cerebrospinal fluid, and urine, and
an agent for measuring the expression level is an antibody or an aptamer bonded to the ADM2 protein;
2) comparing the expression level with that of a normal body weight thyroid cancer patient sample; and
3) determining a higher risk of thyroid cancer exacerbation than that of thyroid cancer patients with normal body weight when the expression level is 1.3 to 1.6 times higher than that of the normal body weight thyroid cancer patient sample,
wherein,
the expression level of the ADM2 protein is determined by an absolute concentration in μg/mL or a difference in relative intensity of a signal.

5. The method of claim 4, wherein, when the expression level is 1.3 to 1.6 times higher than that of the normal body weight thyroid cancer patient sample, the prognosis of the thyroid cancer patient is classified as good prognosis and, in other cases, poor prognosis,
wherein the good prognosis indicates that a disease-free survival rate or survival rate of the thyroid cancer patient after treatment is high and a possibility of treating the thyroid cancer patient is high, and the poor prognosis indicates that the disease-free survival rate or survival rate of the thyroid cancer patient after treatment is low so that there is a high probability of recurrence from the thyroid cancer patient and death due to thyroid cancer.

6. A method of screening a therapeutic agent for thyroid cancer, the method comprising measuring an expression level of ADM2 gene or protein encoded by the gene after administering a candidate substance expected to be capable of curing the thyroid cancer.

7. A kit for diagnosis or prognosis prediction of thyroid cancer, the kit comprising the composition according to claim 1.

8. The kit of claim 7, wherein the kit is an RT-PCR kit, a DNA chip kit or a protein chip kit.
